(19) United States
(12) Patent Application Publication
(10) Pub. No.: US 2024/0102096 A1
(43) Pub. Date: Mar. 28, 2024

(54) METHOD OF TREATMENT WITH TRADIPITANT

(71) Applicant: Vanda Pharmaceuticals Inc., Washington, DC (US)

(72) Inventors: Mihael H. Polymeropoulos, Potomac, MD (US); Sandra P. Smieszek, Cleveland, OH (US)

(21) Appl. No.: 18/448,868

(22) Filed: Aug. 11, 2023

Related U.S. Application Data
(60) Provisional application No. 63/374,984, filed on Sep. 8, 2022.

(51) Int. Cl. C12Q 1/6883; A61K 31/444

(52) U.S. Cl. CPC C12Q 1/6883; A61K 31/444; C12Q 2600/156

(57) ABSTRACT

Disclosed herein is a method of treatment of an individual suffering from gastroparesis or delayed gastric emptying. In an embodiment, the method comprises selecting an individual for treatment with tradipitant based on a presence of a variant in the SLC15A4 gene of the individual, or a biomarker indicative thereof; and administering to the selected individual tradipitant at a dose of 150-400 mg/day.

### CROSS REFERENCE TO RELATED APPLICATIONS

The present patent application claims priority to U.S. Provisional Application No. 63/374,984, filed Sep. 8, 2022.

### BACKGROUND OF THE INVENTION

The application relates generally to use of NK-1 receptor antagonists. More particularly, the application relates the use of the NK-1 antagonist, tradipitant, for treatment of gastroparesis.

Gastroparesis is a serious medical condition characterized by delayed gastric emptying in the absence of mechanical obstruction, and is associated with the symptoms of nausea, vomiting, bloating, fullness after meals, and abdominal pain, along with significant impairment of social and occupational functioning. The estimated prevalence of gastroparesis in the U.S. is over 5 million patients, many of whom remain undiagnosed. Gastroparesis affects women more frequently than men. Common etiologies include diabetes (estimated at >51.7%), post-surgical gastroparesis, and idiopathic gastroparesis (estimated at ˜11.3%).

The only U.S. Food and Drug Administration-approved treatment for gastroparesis is metoclopramide, which was approved in 1979 and carries a black box warning and limitations of use for no more than 3 months due to its potential for severe side effects. Patients are therefore faced with limited therapeutic options. Clinical guidelines recommend, in addition to metoclopramide, the off label use of different drugs including erythromycin, domperidone (not approved in the U.S.), botulinum toxin injections, gastric stimulators, and a variety of surgical procedures in an effort to achieve even temporary relief of some of the symptoms of the disease. Gastroparesis treatment represents a significant unmet medical need, as underscored by the testimonies of interested parties and advocacy organizations including the International Foundation for Gastrointestinal Disorders (IFFGD) and Gastroparesis Patient Association for Cures and Treatments, Inc. (G-Pact).

The precise underlying mechanisms leading to gastroparesis are currently poorly understood and are believed to be diverse in nature. The consensus suggests that gastroparesis arises from a dysregulation of the neuromuscular control of gastric movements that result in the timely emptying of stomach contents. An innate immune dysregulation and injury to the interstitial cells of Cajal (ICC) and other components of the enteric nervous system through paracrine and oxidative stress mediators are believed to be involved in the pathogenesis of gastroparesis. Little is known about the underlying genetic risk factors for gastroparesis.

Two key stimulatory neurotransmitters of the digestive system are acetylcholine and the neuropeptide substance P. Substance P acts by binding the neurokinin 1 receptor (NK-1R) at the gastric neuromuscular junction, and it is believed that there is functional interplay between the acetylcholine and NK-1R systems. Moreover, gastroparesis symptoms are also associated with aberrant physiology of the vagus nerve, which constitutes the major connection between the stomach and the central nervous system. Blockade of the NK-1 receptors may have a dual and potentially therapeutic effect in gastroparesis by affecting gastric motility through local action as well as affecting nausea and vomiting via a direct effect in the brain regions responsible for nausea and vomiting.

Tradipitant is a highly potent, selective, centrally penetrating, and orally active neurokinin-1 (NK-1) receptor antagonist, depicted below as the compound of Formula I:

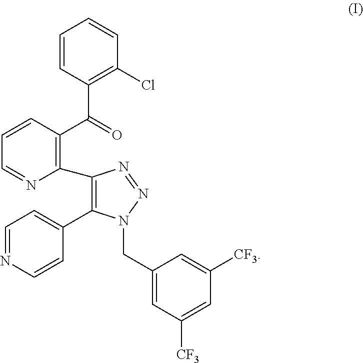 [SMILES: O=C(c1ccccc1Cl)c1cccnc1-c1nnn(Cc2cc(C(F)(F)F)cc(C(F)(F)F)c2)c1-c1ccncc1]

Tradipitant is disclosed in U.S. Pat. No. 7,320,994, and contains six main structural components: the 3,5-bis-trifluoromethylphenyl moiety, two pyridine rings, the triazol ring, the chlorophenyl ring, and the methanone. Tradipitant is known by the chemical names, 2-[1-[[3,5-bis(trifluoromethyl)phenyl]methyl]-5-(4-pyridinyl)-1H-1,2,3-triazol-4-yl]-3-pyridinyl](2-chlorophenyl)-methanone, and {2-[1-(3,5-bistrifluoromethylbenzyl)-5-pyridin-4-yl-1H-[1,2,3]triazol-4-yl]-pyridin-3-yl}-(2-chlorophenyl)-methanone, and has also been known as LY686017 and as VLY-686. U.S. Pat. No. 7,320,994 describes methods for using compounds including tradipitant for treating a condition associated with an excess of tachykinins, most particularly where the condition associated with an excess of tachykinins is depression and anxiety. U.S. Pat. No. 7,320,994 further describes the possibility of using compounds, such as tradipitant, in other such diseases, i.e., because these compounds inhibit the physiological effects associated with an excess of tachykinins. The patent describes the usefulness of such compounds in the treatment of numerous other disorders related to tachykinin receptor activation including psychosis, schizophrenia, and other psychotic disorders; neurodegenerative disorders such as dementia, including senile dementia of the Alzheimer's type, Alzheimer's disease, AIDS-associated dementia, and Down Syndrome; demyelinating diseases such as multiple sclerosis and amyotrophic lateral sclerosis; and other neuropathological disorders, such as peripheral neuropathy, diabetic and chemotherapy-induced neuropathy, and post-herpetic and other neuralgias; acute and chronic obstructive airway diseases such as adult respiratory distress syndrome, bronchopneumonia, bronchospasm, chronic bronchitis, drivercough, and asthma; inflammatory diseases such as inflammatory bowel disease, psoriasis, fibrositis, osteoarthritis, and rheumatoid arthritis; disorders of the musculoskeletal system, such as osteoporosis; allergies such as eczema and rhinitis; hypersensitivity disorders such as poison ivy; ophthalmic diseases such as conjunctivitis, vernal conjunctivitis, and the like; cutaneous diseases such as contact dermatitis, atopic dermatitis, urticaria, and other eczematoid dermatites; addiction disorders such as alcoholism; stress-related somatic disorders; reflex sympathetic dystrophy such as shoulder/hand syndrome; dysthyrnic disorders; adverse immunological reactions such as rejection of transplanted tissues and disorders related to immune enhancement or suppression such as systemic lupus erythematosis; gastrointestinal disorders or diseases associated with the neuronal control of viscera such as ulcerative colitis, Crohn's disease, and irritable bowel syndrome; disorders of bladder function such as bladder detrusor hyper-reflexia and incontinence; atherosclerosis; fibrosin and collagen diseases such as scleroderma and eosinophilic fascioliasis; irritative symptoms of benign prostatic hypertrophy; disorders associated with blood pressure, such as hypertension; or disorders of blood flow caused by vasodilation and vasospastic diseases, such as angina, migraine, and Reynaud's disease; emesis, including chemotherapy-induced nausea and emesis; and pain or nociception, for example, that are attributable to or associated with any of the foregoing conditions. Finally, the patent describes that such compounds are effective in amounts to be determined, ranging from 0.001 mg/kg/day to 100 mg/kg/day.

Tradipitant is known to be therapeutically administered through a variety of routes of administration by which it is bioavailable. U.S. Pat. No. 7,320,994 discloses administration of tradipitant by oral and parenteral routes, e.g., orally, by inhalation, subcutaneously, intramuscularly, intravenously, transdermally, intranasally, rectally, occularly, topically, sublingually, and buccally, with oral administration being generally preferred for treatment.

Crystalline Forms IV and V of tradipitant are disclosed in U.S. Pat. No. 7,381,826, and a process for preparing crystalline {2-[1-(3,5-bistrifluoromethylbenzyl)-5-pyridin-4-yl-1H-[1,2,3]triazol-4-yl]-pyridin-3-yl}-(2-chlorophenyl)-methanone, Form IV is disclosed in U.S. Pat. Nos. 8,772,496 and 9,708,291.

Additionally, methods of treatment of gastroparesis and gastric motility disorders are disclosed in International Patent Application Publication Nos. WO 2019/099883 and WO 2020/117811, each of which is incorporated herein by reference.

### BRIEF DESCRIPTION OF THE INVENTION

Various aspects of the invention disclosed herein relate to methods of treatment with tradipitant. One such method includes selecting an individual for treatment with tradipitant based on a biomarker which indicates a presence of a variant in the Solute carrier family 15 A4 (SLC15A4) gene of the individual; and administering tradipitant to the selected individual. In certain embodiments, the tradipitant may be administered at a dose of 150-400 mg/day, 150-300 mg/day, 150-200 mg/day, about 170 mg/day, or 85 mg twice daily (bid), in an immediate release form containing tradipitant and at least one pharmaceutically acceptable excipient. The immediate release form may be, e.g., a capsule or a tablet for oral administration.

In certain embodiments, the selecting comprises determining that the individual suffers from gastroparesis, or more particularly, from idiopathic gastroparesis. In other embodiments, the individual may suffer from diabetic gastroparesis or post-surgical gastroparesis. In certain embodiments, the selecting further comprises determining that the individual suffers from delayed gastric emptying.

In certain embodiments, the selecting further comprises determining the presence or absence of the variant in the SLC15A4 gene of the individual. This may be accomplished by obtaining or having obtained a biological sample from the patient; and performing or having performed a genotyping assay on the biological sample to determine if the individual has a SLC15A4 gene variant genotype. Performing the genotyping assay may include extracting or having extracted genomic DNA or mRNA from the biological sample, and sequencing or having sequenced SLC15A4 DNA derived from the extracted genomic DNA or from the extracted mRNA. The step of sequencing or having sequenced the DNA includes amplifying or having amplified a SLC15A4 region in the extracted genomic DNA or mRNA to prepare a DNA sample enriched in DNA from the SLC15A4 gene region; and sequencing or having sequenced the DNA sample by hybridizing the DNA sample to nucleic acid probes to determine if the patient has a SLC15A4 variant genotype.

In certain embodiments, the variant in the SLC15A4 gene that is detected in the selected individual is a missense variant, and may particularly be SLC15A4:NM145648:exon2:c.T716C:p.V239A, rs33990080.

In certain embodiments, a biological sample taken from the selected individual may show a greater proportion of pro-inflammatory M1 macrophages, and/or a lower proportion of anti-inflammatory M2 macrophages, than is observed in individuals having a wild type SLC15A4 genotype.

These and other aspects, advantages and salient features of the invention will become apparent from the following detailed description, which, when taken in conjunction with the annexed drawings, disclose embodiments of the invention.

### BRIEF DESCRIPTION OF THE DRAWINGS

FIG. 1A shows a Manhattan plot showing top results for GWAS: idiopathic gastroparesis versus controls.

FIG. 1B shows a Manhattan plot for GWAS: diabetic gastroparesis cases versus controls.

FIG. 2 is a Zoom plot showing SLC15A4 rs33990080 variant of interest detected in the idiopathic versus controls analysis.

It is noted that the drawings are intended to depict only typical aspects of the disclosure, and therefore should not be considered as limiting the scope of the disclosure.

### DETAILED DESCRIPTION OF THE INVENTION

At least one embodiment of the present invention is described below in reference to its application in connection with the use of tradipitant for the treatment of gastroparesis. Although some embodiments of the invention are illustrated relative to a specific disorder, i.e., gastroparesis, it is understood that the teachings are equally applicable to symptoms associated with gastroparesis, which may include nausea, vomiting, early satiety, postprandial fullness, dyspepsia (indigestion), functional dyspepsia, bloating, and abdominal pain. The teachings are further applicable to both individuals who have and who have not been diagnosed with gastroparesis, as well as those suffering from delayed gastric emptying. Further, the individual may suffer from idiopathic gastroparesis, post-operative gastroparesis, or diabetic gastroparesis.

Individuals suffering from gastroparesis can be treated by orally administering tradipitant in an effective amount. As used herein, the term “effective amount” of tradipitant refers to an amount that is effective in treating the disorders described herein, and may refer to an amount in conjunction with a dosing frequency required to achieve plasma concentrations of at least about 100 ng/mL, e.g., 125 ng/mL or greater, 150 ng/mL or greater, 175 ng/mL or greater, 200 ng/mL or greater, or 225 ng/mL or greater. Such plasma concentration levels can be achieved, e.g., by orally administering to the patient tradipitant in a solid immediate release form comprising one or more pharmaceutically acceptable excipients and tradipitant in an amount of, e.g., 100 to 400 mg/day, 100 to 300 mg/day, 100 to 200 mg/day, or about 85-170 mg/day, which may be administered as, e.g., 50 to 200 mg bid, 50 to 150 mg bid, 50 to 100 mg bid, or about 85 mg bid, or about 150 to 400 mg/day, 150 to 300 mg/day, or about 150 to 200 mg/day, which may be administered as, e.g., 75 to 200 mg bid, 75 to 150 mg bid, or 75 to 100 mg bid, or about 85 mg bid, as described in PCT publication WO 2016/141341 A1. With regard to dosing, “qd” refers to dosing once per day; “bid” dosing typically means dosing once in the morning and once in the evening, generally no less than about 8 hours or more than about 16 hours apart, e.g., 10 to 14 hours or 12 hours (Q12H).

As used herein, the terms “patient,” “subject,” and “individual” refer to human beings, as well as companion animals (e.g., dogs and cats) and other domesticated animals (e.g., horses, cattle, and sheep). It will be understood that the most preferred patient is a human being.

The invention further relates to the treatment of gastroparesis or delayed gastric emptying, either prophylactically or therapeutically. Further, the terms “treatment” and “treating” are intended to refer to all processes wherein there may be a slowing, interrupting, arresting, controlling, or stopping of the progression of the disorders described herein, and is intended to include prophylactic treatment of such disorders, but does not necessarily indicate a total elimination of all disorder symptoms.

In one embodiment, a method is provided for treating an individual as described herein. According to the method, the individual may be selected for treatment with tradipitant based on a determination that the individual's genotype includes a variant in the SLC15A4 gene. The variant may be detected in a biological sample taken from the patient. The individual may further be selected on the basis of a medical professional's conclusion that the individual suffers from gastroparesis, particularly idiopathic gastroparesis, or delayed gastric emptying.

As noted, the individual selection process may include determining the presence or absence of the variant in the SLC15A4 gene of the individual. In certain embodiments, such a determination may be made by obtaining or having obtained a biological sample from the patient; and performing or having performed a genotyping assay on the biological sample to determine if the individual has a SLC15A4 gene variant genotype. In this context, “obtaining” may refer to collecting or acquiring a biological sample from the patient, while “having obtained” may refer to referring, instructing, or otherwise causing another individual, e.g., a medical or healthcare professional, to perform the obtaining. “Having obtained” may also refer to having previously caused the obtaining to have been performed, e.g., where an assay that identifies the individual's SLC15A4 genotype has been performed in the past, and the result may be reviewed in the individual's medical records. Similarly, in this context, “performing the genotyping assay” may refer to physically performing the steps to examine the individual's DNA using a genotyping assay, while “having performed” may refer to referring, instructing, or otherwise causing another individual, e.g., a medical or healthcare professional, to carry out the performing. The expression, “having performed” may also refer to having previously caused the performance of the assay. Performing (or having performed) the assay may include the steps of extracting or having extracted genomic DNA or mRNA from the biological sample, and sequencing or having sequenced SLC15A4 DNA derived from the extracted genomic DNA or from the extracted mRNA. The sequencing (or having sequenced) step may further comprise amplifying or having amplified a SLC15A4 region in the extracted genomic DNA or mRNA to prepare a DNA sample enriched in DNA from the SLC15A4 gene region; and sequencing (or having sequenced) the DNA sample by hybridizing the DNA sample to nucleic acid probes to determine if the patient has a SLC15A4 variant genotype.

The SLC15A4 locus is polymorphic. In certain embodiments, the SLC15A4 variant detected in a selected individual may be a missense variant, and may particularly be SLC15A4:NM145648:exon2:c.T716C:p.V239A, rs33990080. Other variants will be known and understood by one of skill in the art.

Because SLC15A4 has been shown to mediate M1-prone metabolic shifts, the individual's SLC15A4 genotype may also be determined based on an inference as to the individual's SLC15A4 genotype made from the relative proportion of M1- and M2-macrophages present in a biological sample taken from the individual. SLC has been shown to mediate Nil-prone metabolic shifts in macrophages and guard immune cells from metabolic stress. When the anti-inflammatory M2 macrophages are switched to pro-inflammatory M1 macrophages, delayed gastric emptying may result. Therefore, the presence of a greater proportion of pro-inflammatory M1 macrophages than is observed in an individual having a wild type SLC15A4 genotype may be considered a biomarker consistent with a SLC15A4 variant genotype. Similarly, the presence of a lower proportion of anti-inflammatory M2 macrophages than is observed in an individual having a wild type SLC15A4 genotype, may also be considered a biomarker consistent with a SLC15A4 variant genotype. Such biomarkers may be employed in the method described herein in place of direct determination of the individual's genotype.

In the event that the individual has a wild type genotype at the SLC15A4 locus, and/or a biomarker consistent with such a genotype, the individual may not be selected for treatment, or may be selected for a treatment that differs in one or more of the active pharmaceutical ingredient, the dose selected, or other features of the treatment regimen. On the other hand, in the event that the individual has a SLC15A4 variant genotype, i.e. a non-wild type genotype at the SLC15A4 locus, and/or a biomarker consistent therewith, the individual may be selected for treatment according to further steps of the method described herein.

Following selection of the individual for treatment as described herein, the method may further include administering to the selected individual tradipitant at a dose effective to treat the individual's symptoms of gastroparesis. Exemplary effective doses may be, e.g., 150-400 mg/day, 100-400 mg/day, 150 to 300 mg/day, 100-300 mg/day, 150 to 200 mg/day, 100-200 mg/day, or about 170 mg/day. The dosage amount may be administered as, e.g., 75 to 200 mg bid, 50-200 mg bid, 75 to 150 mg bid, 50-150 mg bid, 75 to 100 mg bid, 50-100 mg bid, or about 85 mg bid. As described herein, the dose administered may be sufficient to achieve and maintain a plasma concentration level of tradipitant in the individual of at least about 100 ng/mL, e.g., 125 ng/mL or greater, 150 ng/mL or greater, 175 ng/mL or greater, 200 ng/mL or greater, or 225 ng/mL or greater.

### Example 1: Genome-Wide Association Study (GWAS)

A genome-wide association study is performed, comparing idiopathic and diabetic gastroparesis cases with controls, and contrasting idiopathic gastroparesis cases with diabetic gastroparesis cases, to ascertain genetic risk factors for gastroparesis. Samples are obtained from highly phenotyped gastroparesis studies of both diabetic and idiopathic gastroparesis.

Methods

Whole-genome sequencing is performed on n=1385 samples obtained from consenting gastroparesis patients participating in one of two gastroparesis studies: a phase II clinical study (VP-VLY-686-2301 and an ongoing phase III gastroparesis clinical study (VP-VLY-686-3301). The phase II study cohort (n=119) is composed of 90.7% females; the average age is 45.9; 86.2% of the patients self-identify as White, 10.5% self-report as Black or African American, 2% self-report as American Indian or Alaska Native, and 1.3% report as Asian. Inclusion criteria for this study include patients with idiopathic or diabetic gastroparesis with moderate to severe nausea, delayed gastric emptying, daily 50% worst average nausea score ≥3, and GCSI nausea score ≥3 at screening. In the phase III study, subjects include male and female adults ages 18-70 with a diagnosis of diabetic or idiopathic gastroparesis. This cohort is also multiethnic, and includes 70.2% females. The average age is 48.5; 64.5% of the patients self-identify as White, 31.7% self-report as Black or African American, 0.8% self-report as American Indian or Alaska Native, and 1.9% report as Asian. Subjects have evidence of delayed gastric emptying, and moderate to severe nausea, daily average nausea score of >2.5, at least one episode of vomiting, PAGI-SYM nausea score of ≥2 at screening, and controlled blood glucose levels, with HbA1c<10%. Self-reported ancestry is further confirmed with PCA.

DNA samples are quantified using fluorescent-based assays (PicoGreen) to accurately determine whether sufficient material is available for library preparation and sequencing. DNA sample size distributions are profiled by a Fragment Analyzer (Advanced Analytics) or BioAnalyzer (Agilent Technologies), to assess sample quality and integrity. WGS libraries are prepared using the Truseq DNA PCR-free Library Preparation Kit. Whole Genome data are processed on NYGC automated pipeline. Paired-end 150 bp reads are aligned to the GRCh37 human reference (BWA-MEM v0.7.8) and processed with GATK best-practices workflow (GATK v3.4.0). The mean coverage is 35.8, reflecting the average of the samples. All high-quality variants obtained from GATK pipeline are functionally annotated (intronic, intergenic, splicing, nonsynonymous, stopgain and frameshifts) based on RefSeq transcripts. Cases and controls are called with the sample pipeline, only variants passing GATK's Variant Quality Score Recalibration (VQSR) internal minor allele frequency (MAF) cutoff are included in the analysis.

A GWAS is performed using variants with minor allele frequencies (MAFs) higher than 1% and allele count of 10 or more, with the same analysis design used in the landmark COVID-19 HGI GWAS (Initiative C-19 HG. Mapping the human genetic architecture of COVID-19. Nature. 2021. doi:10.1038/s41586-021-03767-x). Single variant association tests are performed using a GWAS additive model framework. Logistic models adjusted for PC, age, and sex are conducted in PLINK. The analysis is limited to individuals of European (EUR) genetic ancestry due to the largest cohort available in this ancestry, and in order to avoid confounders in the form of population substructure. The Principal Component Analysis and PC definitions are based on that provided by the COVID-19 Host Genetics Initiative, available here for reference: https://github.com/covid19-hg/pca_projection. A common set of SNPs is used for ancestry definitions to match those for 1000 genomes project (V3)

Results

Results are obtained from logistic models for the 4 following GWAS analyses: 1) idiopathic vs. controls, 2) diabetic vs. controls, 3) gastroparesis cases vs. non-gastroparesis matched controls and 4) diabetic vs. idiopathic cases.

Idiopathic Gastroparesis

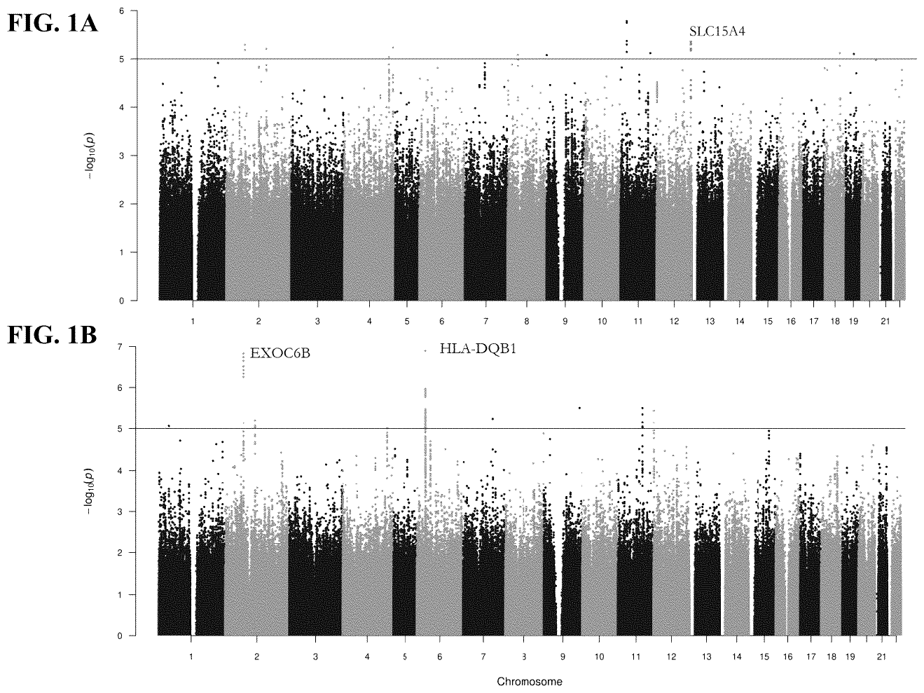

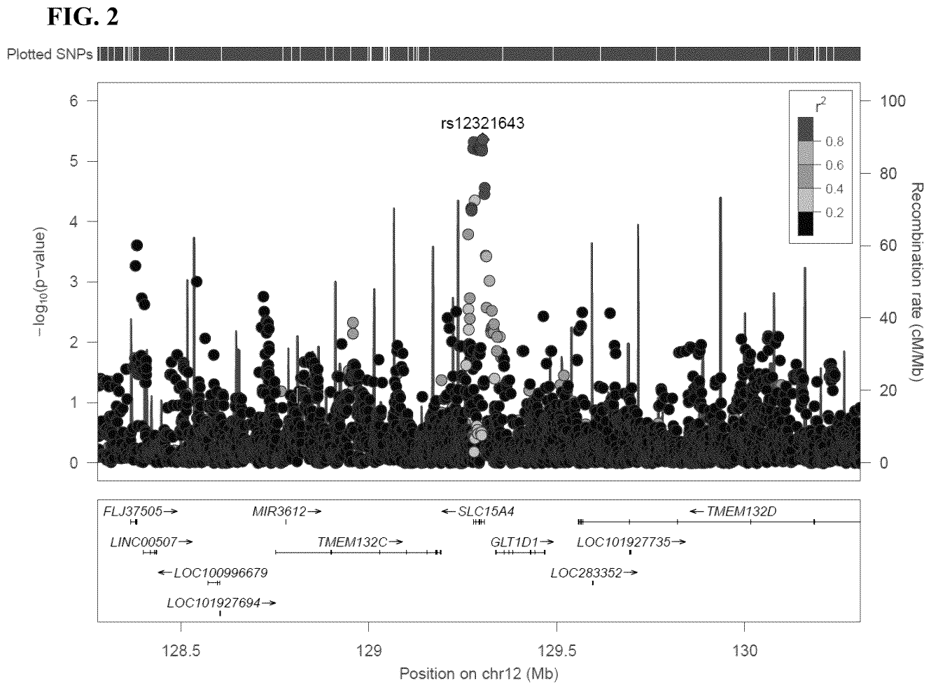

In order to avoid confounders, the largest set of EUR ancestry is used for the purpose of the idiopathic and diabetic analyses. The analysis consists of variants with MAF>1, EUR individuals (PCA-defined) in both cases (n=319) and controls (n=896). For comparisons of variant frequencies, both gastroparesis cohorts are used, controls and gnomAD⁷ (matched by ancestry as defined by PCA). Amongst the top strongest loci is a signal from a variant within the Solute carrier family 15 (SLC15A4) gene: lead variant rs10847696 (p-value<0.000004 (OR:1.9)). The ns variant of interest is also depicted in the Manhattan plot in FIG. 1A, and the SLC15A4 regional zoom plot in FIG. 2. The top non-synonymous variant is SLC15A4:NM145648:exon2:c.T716C:p.V239A, rs33990080.

Altogether, 69 carriers are observed among 214 idiopathic patients, vs. 165 out of 896 ancestry-matched controls (MAF: cases 0.18; MAF: controls 0.09) and OR: 1.9. The MAF in gastroparesis (diabetic and idiopathic patients combined) is 0.16. The global MAF among non-Finnish-Europeans (NFE) in a public database (gnomAD) is 0.09. The significant effect persists when idiopathic cases are compared with diabetic cases, as the MAF of diabetic cases (0.10) is comparable to that of population controls (0.09). The variant is a highly statistically significant (p-value<10⁻¹⁷) eQTL for chr12 GLT1D1 and SLC15A4 (GTEX). The identified variant carriers have a significantly higher nausea score at baseline with a mean difference ˜0.3 unit, and ˜0.6 if comparing homozygotes (p-value<0.04). There are additional other significant variants reported within the SLC15A4 gene in association with idiopathic gastroparesis, further supporting the effect carried by the identified region. The top results for the idiopathic vs. controls analysis are presented in Table 1.

TABLE 1
Top GWAS variants for idiopathic cases versus controls

| | | | | | | | | OR.idio. |
|---|---|---|---|---|---|---|---|---|
| chr | bp | Ref | Alt | Type | Nearest Gene | AAChange | dbSNP | vs.cont |
| 2 | 70575204 | A | C | intergenic | FAM136A, TGFA | — | rs10203624 | 0.51 |
| 2 | 150783253 | G | A | intergenic | LINC01931, LINC01817 | — | rs11689526 | 0.57 |
| 4 | 168501627 | G | T | intergenic | SPOCK3, ANXA10 | — | rs1904115 | 1.67 |
| 8 | 40159237 | T | C | intergenic | C8orf4, ZMAT4 | — | rs16889111 | 2.58 |
| 9 | 1320866 | A | G | intergenic | DMRT2, SMARCA2 | — | rs72687269 | 4.23 |
| 11 | 24392805 | C | G | intergenic | MIR8054, LUZP2 | — | rs2403906 | 1.89 |
| 12 | 129297153 | G | A | intronic | SLC15A4 | — | rs10847696 | 1.99 |
| 12 | 129299446 | A | G | exonic | SLCI5A4 | NM_145648 [symbol] exon2: | rs33990080 | 2.01 |
| | | | | | | c.T716C:p.V239A | | |
| 18 | 57237671 | C | T | intronic | CCBE1 | — | rs7227690 | 3.43 |
| 19 | 31224845 | C | T | intergenic | ZNF536, LINC01791 | — | rs142557503 | 6.28 |

| | | | | | | gnomAD_ | gnomAD_ |
|---|---|---|---|---|---|---|---|
| | P.idio. | MAF. | MAF. | MAF. | MAF. | genome_ | genome_ |
| chr | vs.cont | diab | idio | gastro.alt | nongastro | ALL | NFE |
| 2 | 5.08E−06 | 0.200 | 0.154 | 0.169 | 0.260 | 0.318 | 0.265 |
| 2 | 6.00E−06 | 0.400 | 0.273 | 0.314 | 0.385 | 0.316 | 0.377 |
| 4 | 9.16E−06 | 0.171 | 0.341 | 0.285 | 0.230 | 0.229 | 0.239 |
| 8 | 8.28E−06 | 0.033 | 0.093 | 0.074 | 0.040 | 0.053 | 0.047 |
| 9 | 8.37E−06 | 0.010 | 0.047 | 0.034 | 0.013 | 0.010 | 0.016 |
| 11 | 1.14E−06 | 0.186 | 0.269 | 0.241 | 0.166 | 0.210 | 0.211 |
| 12 | 4.13E−06 | 0.114 | 0.189 | 0.165 | 0.101 | 0.092 | 0.092 |
| 12 | 4.81E−06 | 0.100 | 0.182 | 0.155 | 0.096 | 0.065 | 0.089 |
| 18 | 7.60E−06 | 0.024 | 0.061 | 0.049 | 0.021 | 0.037 | 0.022 |
| 19 | 7.92E−06 | 0.029 | 0.035 | 0.033 | 0.006 | 0.009 | 0.015 |

[symbol]

indicates data missing or illegible when filed

Other identified loci include intergenic variants in the region of MIR8054 and LUZP2 genes as well as other loci depicted in Table 1. The effect of this SLC15A4 variant is replicated in another batch of EUR gastroparesis samples with OR 1.6, p-value<10⁻⁵.

Diabetic vs. Idiopathic Gastroparesis

In this analysis set, idiopathic controls are super controls, as this is a set with a confirmed negative diabetes diagnosis. Top variants in diabetic gastroparesis GWAS include variants pointing to HLA-DQB1, with lead variant rs9275638. Also among the top variants is rs9273363, which is a known risk factor for Type 1 diabetes, with an OR of 2.1, p-value<10⁻⁵. rs9273363 is a common variant with a MAF of 0.39 in diabetic gastroparesis cases versus MAF of 0.22 in idiopathic controls, as well as MAF 0.27 in matched EUR controls. The variant is tagging the HLA DQB1*03:02 haplotype and the 6q22.33 region, which contains the genes encoding protein tyrosine phosphatase receptor k (PTPRK). Amongst the other top variants are loci within EXOC6B depicted in FIG. 1B and Table 2.

TABLE 2
Top GWAS variants for idiopathic cases versus controls

| | | | | | | | | OR.logit.diab. |
|---|---|---|---|---|---|---|---|---|
| chr | bp | Ref | Alt | Type | Nearest Gene | ExonitFunt | dbSNP | vs.id [symbol] |
| 2 | 72416952 | TCTC | — | intronic | EXOC6B | — | rs139115191 | 3.36 |
| 2 | 14998082 | G | C | intergenic | LINCO1191, DPP10 | — | rs31372089 | 3.34 |
| 6 | 32698988 | A | C | intergenic | HLA-DQB1, HLA-DQA2 | — | rs7751856 | 3.03 |
| 6 | 32729642 | C | G | exonic | HLA-DQB2 | synonymous SNV | rs762815 | 2.5 |
| 11 | 95405933 | C | T | intergenic | LOC100129203, | — | rs7931781 | 2.40 |
| | | | | | FAM76B | | | |
| 12 | 2964735 | G | A | ncRNA_ | LOC100507424 | — | rs7138715 | 2.86 |
| | | | | Intronic | | | | |
| 15 | 78567853 | G | T | exonic | DNAJA4 | synonymous SNV | rs967821 | 2.79 |

| | | | | | | gnomAD_ | gnomAD_ |
|---|---|---|---|---|---|---|---|
| | P.logit.diab. | MAF. | MAF. | MAF. | MAF. | genome_ | genome_ |
| chr | vs.id [symbol] | diab | idio | gastro.alt | nongastro | ALL | NFE |
| 2 | 1.45E−07 | 0.291 | 0.122 | 0.177 | 0.184 | 0.115 | 0.164 |
| 2 | 6.09E−06 | 0.229 | 0.096 | 0.140 | 0.120 | 0.090 | 0.129 |
| 6 | 1.26E−07 | 0.610 | 0.435 | 0.492 | 0.462 | 0.489 | 0.540 |
| 6 | 3.45E−06 | 0.55 | 0.4 | 0.45 | 0.41 | 0.66 | 0.57 |
| 11 | 3.08E−06 | 0.595 | 0.409 | 0.470 | 0.479 | 0.561 | 0.538 |
| 12 | 3.55E−05 | 0.286 | 0.143 | 0.190 | 0.191 | 0.263 | 0.176 |
| 15 | 1.15E−05 | 0.286 | 0.150 | 0.194 | 0.175 | 0.212 | 0.193 |

[symbol]

indicates data missing or illegible when filed

EXOC6B's top variant is a UTR variant, rs41416, which is highly increased among diabetic but not idiopathic cases (OR 3.3, p-value<10⁻⁷). This exocyst complex gene is associated with increased risk of Type II diabetes.

Polygenic scores are constructed for diabetes by summing the number of risk alleles carried by each individual, weighted by the effect size estimates from well-established genome-wide significant associations derived from Li et al., 2021 (60 SNPs). Idiopathic gastroparesis differentiates from diabetic gastroparesis with a diabetes genetic risk score, with a statistically significant result (p-value<0.0004). This reinforces the capacity to subgroup cases based on diabetes risk score. Additionally, the frequencies of previously reported variants are explored in a functional dyspepsia GWAS. Two variants previously reported come out as nominally significant: rs2595968 (NFASC p-value<0.004) and rs17597505 (p-value<0.03).

### Discussion

Whole-genome sequencing provides unique insights into genetic determinants of gastroparesis, by uncovering associations between common as well as rare genetic variants. Such loci can be extremely helpful, because they are often coding, thereby pointing directly to a causal gene. In this study focused primarily on idiopathic and diabetic gastroparesis cases, novel variants are associated with idiopathic gastroparesis, as well as known risk factors previously associated with diabetes. A novel genetic association of the SLC15A4 (p.Val239A1a) risk variant confers a greater risk of idiopathic gastroparesis.

SLC15A4 is a lysosome-resident amino acid/oligopeptide transporter that is preferentially expressed in immune cells. This proton-coupled amino-acid transporter mediates the transmembrane transport of L-histidine and some di- and tripeptides from inside the lysosome to the cytosol, and plays a key role in innate immune responses. SLC15A4 is involved in TLR7/9-dependent type I interferon production, and is associated with disorders such as inflammatory bowel diseases and systemic lupus erythematosus. High levels of SLC15A4 transcripts are observed in human antigen-presenting cells, including dendritic cells, activated macrophages, and B cells. SLC15A4 is shown to mediate M1-prone metabolic shifts in macrophages and guard immune cells from metabolic stress. SLC15A4 loss disturbs the coupling of glycolysis and the TCA cycle, and SLC15A4-deficient macrophages prefer to use glutamine rather than glucose as a carbon source for the TCA cycle. SLC15A4-deficient macrophages produce low levels of itaconate and proinflammatory IL-12 cytokine members. Correspondingly, SLC15A4^{-/-} mice develop a less severe form of Th1-dependent colitis than SLC15A4^{+/+} mice. SLC15A4 is shown to promote colitis through Toll-like receptor 9 and NODI-dependent innate immune responses. It is involved in maintaining the histidine homeostasis within intracellular compartments and eliciting effective innate immune responses. SLC15A4-intact but not SLC15A4-deficient macrophages become resistant to fluctuations in environmental nutrient levels by limiting the use of the glutamine source; thus, SLC15A4 is critical for macrophage's respiratory homeostasis.

When the anti-inflammatory M2 macrophages switch to pro-inflammatory M1 macrophages, delayed gastric emptying is evident in animal models. This is consistent with the GWAS findings. These findings suggest a mechanism of metabolic regulation in which an amino acid transporter acts as a gatekeeper that protects immune cells' ability to acquire an M1-prone metabolic phenotype in inflammatory tissues by mitigating metabolic stress. The detected loci may be directly connected to macrophage polarization either via gain of function or hyperexpression.

Idiopathic gastroparesis is associated with a reduction in both myenteric and intramuscular interstitial cells of Cajal. Successful gastric emptying results from the successful cooperation between smooth muscle, enteric and autonomic nerves, and interstitial cells of Cajal (ICC). Multiple paths may lead to the same aberrent pathway, e.g., the M1/M2 macrophage polarization may lead to gastroparesis in both the diabetic and idiopathic etiologies of gastroparesis. In particular, macrophage polarization may lead to the destruction if the interstitial cells of Cajal, effectively leading to abnormal gastric emptying. The present example shows potential risk factors from a genetic perspective.

Other variants delineated in this study call for further confirmation and research into their mechanisms of action. By depleting the diabetic cases and doing a sub-analysis, a homogenous set is created for exploratory GWAS. Risk is successfully detected for HLA-DQB1 in the diabetes cohort, and other loci are identified that call for further confirmatory studies, e.g., EXOC6B.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed, but that the invention will include all embodiments falling within the scope of the appended claims.

Embodiments of the present disclosure may include the following features:
Embodiment 1. A method comprising: selecting an individual for treatment with tradipitant based on a presence of a variant in the SLC15A4 gene of the individual; and administering to the individual tradipitant at a dose of 150-400 mg/day.
Embodiment 2. The method of embodiment 1, wherein the dose of tradipitant is 150-300 mg/day.
Embodiment 3. The method of embodiment 2, wherein the dose of tradipitant is 150-200 mg/day.
Embodiment 4. The method of embodiment 3, wherein the dose of tradipitant is 170 mg/day.
Embodiment 5. The method of embodiment 4, wherein the dose of tradipitant is 85 mg twice daily (bid).
Embodiment 6. The method of any one of embodiments 1-5, wherein the variant in the SLC15A4 gene is a missense variant.
Embodiment 7. The method of embodiment 6, wherein the variant in the SLC15A4 gene is SLC15A4:NM145648:exon2:c.T716C:p.V239A, rs33990080.
Embodiment 8. The method of any one of embodiments 1-7, wherein the individual has a greater proportion of pro-inflammatory M1 macrophages than is observed in an individual having a wild type SLC15A4 genotype.
Embodiment 9. The method of any one of embodiments 1-7, wherein the individual has a lower proportion of anti-inflammatory M2 macrophages than is observed in an individual having a wild type SLC15A4 genotype.
Embodiment 10. The method of any one of embodiments 1-5, wherein the selecting further comprises determining that the individual suffers from gastroparesis.
Embodiment 11. The method of embodiment 10, wherein the gastroparesis further comprises idiopathic gastroparesis.
Embodiment 12. The method of any one of embodiments 1-5, wherein the selecting further comprises determining that the individual suffers from delayed or abnormal gastric emptying.
Embodiment 13. The method of any one of embodiments 1-5, wherein the selecting further comprises: determining the presence or absence of the variant in the SLC15A4 gene of the individual by: obtaining or having obtained a biological sample from the patient; and performing or having performed a genotyping assay on the biological sample to determine if the individual has a SLC15A4 gene variant genotype.
Embodiment 14. The method of embodiment 13, wherein the performing or having performed the genotyping assay step comprises: extracting or having extracted genomic DNA or mRNA from the biological sample, and sequencing or having sequenced SLC15A4 DNA derived from the extracted genomic DNA or from the extracted mRNA, wherein the sequencing or having sequenced step further comprises: amplifying or having amplified a SLC15A4 region in the extracted genomic DNA or mRNA to prepare a DNA sample enriched in DNA from the SLC15A4 gene region; and sequencing or having sequenced the DNA sample by hybridizing the DNA sample to nucleic acid probes to determine if the patient has a SLC15A4 variant genotype.
Embodiment 15. A method comprising: selecting an individual for treatment with tradipitant based on a presence of a biomarker in a biological sample taken from the individual, wherein the biomarker is consistent with a SLC15A4 variant genotype; and administering to the individual tradipitant at a dose of 150-400 mg/day.
Embodiment 16. The method of embodiment 15, wherein the biomarker includes a greater proportion of pro-inflammatory M1 macrophages than is observed in an individual having a wild type SLC15A4 genotype.
Embodiment 17. The method of embodiment 15, wherein the biomarker includes a lower proportion of anti-inflammatory M2 macrophages than is observed in an individual having a wild type SLC15A4 genotype.
Embodiment 18. The method of embodiment 15, wherein the dose of tradipitant is 150-300 mg/day.
Embodiment 19. The method of embodiment 18, wherein the dose of tradipitant is 150-200 mg/day.
Embodiment 20. The method of embodiment 19, wherein the dose of tradipitant is 170 mg/day.
Embodiment 21. The method of embodiment 20, wherein the dose of tradipitant is 85 mg twice daily (bid).
Embodiment 22. The method of any one of embodiments 15-21, wherein the selecting further comprises determining that the individual suffers from gastroparesis.
Embodiment 23. The method of embodiment 22, wherein the gastroparesis further comprises idiopathic gastroparesis.
Embodiment 24. The method of any one of embodiments 15-21, wherein the selecting further comprises determining that the individual suffers from delayed or abnormal gastric emptying.

What is claimed is:

1. A method comprising:
selecting an individual for treatment with tradipitant based on a presence of a variant in the SLC15A4 gene of the individual; and
administering to the individual tradipitant at a dose of 150-400 mg/day.

2. The method of claim 1, wherein the dose of tradipitant is 150-300 mg/day.

3. The method of claim 2, wherein the dose of tradipitant is 150-200 mg/day.

4. The method of claim 3, wherein the dose of tradipitant is 170 mg/day.

5. The method of claim 4, wherein the dose of tradipitant is 85 mg twice daily (bid).

6. The method of claim 1, wherein the variant in the SLC15A4 gene is a missense variant.

7. The method of claim 6, wherein the variant in the SLC15A4 gene is SLC15A4:NM145648:exon2:c.T716C:p.V239A, rs33990080.

8. The method of claim 1, wherein the individual has:
a greater proportion of pro-inflammatory M1 macrophages than is observed in an individual having a wild type SLC15A4 genotype,
a lower proportion of anti-inflammatory M2 macrophages than is observed in the individual having the wild type SLC15A4 genotype, or
the greater proportion of pro-inflammatory M1 macrophages than is observed in the individual having the wild type SLC15A4 genotype and the lower proportion of anti-inflammatory M2 macrophages than is observed in the individual having the wild type SLC15A4 genotype.

9. The method of claim 1, wherein the selecting further comprises:
determining that the individual suffers from gastroparesis,
wherein the gastroparesis further comprises idiopathic gastroparesis.

10. The method of claim 1, wherein the selecting further comprises determining that the individual suffers from delayed or abnormal gastric emptying.

11. The method of claim 1, wherein the selecting further comprises:
determining the presence or absence of the variant in the SLC15A4 gene of the individual by:
obtaining or having obtained a biological sample from the patient; and
performing or having performed a genotyping assay on the biological sample to determine if the individual has a SLC15A4 gene variant genotype.

12. The method of claim 11, wherein the performing or having performed the genotyping assay step comprises:
extracting or having extracted genomic DNA or mRNA from the biological sample, and
sequencing or having sequenced SLC15A4 DNA derived from the extracted genomic DNA or from the extracted mRNA,
wherein the sequencing or having sequenced step further comprises: amplifying or having amplified a SLC15A4 region in the extracted genomic DNA or mRNA to prepare a DNA sample enriched in DNA from the SLC15A4 gene region; and
sequencing or having sequenced the DNA sample by hybridizing the DNA sample to nucleic acid probes to determine if the patient has a SLC15A4 variant genotype.

13. A method comprising:
selecting an individual for treatment with tradipitant based on a presence of a biomarker in a biological sample taken from the individual, wherein the biomarker is consistent with a SLC15A4 variant genotype; and
administering to the individual tradipitant at a dose of 150-400 mg/day.

14. The method of claim 13, wherein the biomarker includes:
a greater proportion of pro-inflammatory M1 macrophages than is observed in an individual having a wild type SLC15A4 genotype,
a lower proportion of anti-inflammatory M2 macrophages than is observed in the individual having the wild type SLC15A4 genotype, or
the greater proportion of pro-inflammatory M1 macrophages than is observed in the individual having the wild type SLC15A4 genotype and the lower proportion of anti-inflammatory M2 macrophages than is observed in the individual having the wild type SLC15A4 genotype.

15. The method of claim 13, wherein the dose of tradipitant is 150-300 mg/day.

16. The method of claim 15, wherein the dose of tradipitant is 150-200 mg/day.

17. The method of claim 16, wherein the dose of tradipitant is 170 mg/day.

18. The method of claim 17, wherein the dose of tradipitant is 85 mg twice daily (bid).

19. The method of claim 13, wherein the selecting further comprises:
determining that the individual suffers from gastroparesis,
wherein the gastroparesis further comprises idiopathic gastroparesis.

20. The method of claim 13, wherein the selecting further comprises determining that the individual suffers from delayed or abnormal gastric emptying.
